(19) United States
(12) Patent Application Publication
(10) Pub. No.: US 2016/0250470 A1
(43) Pub. Date: Sep. 1, 2016

(54) Rehabilitation Monitor And Pain Treatment Assembly

(71) Applicant: Stryker Corporation, Kalamazoo, MI (US)

(72) Inventor: Donald W. Malackowski, Schoolcraft, MI (US)

(73) Assignee: Stryker Corporation, Kalamazoo, MI (US)

(21) Appl. No.: 15/050,845

(22) Filed: Feb. 23, 2016

Related U.S. Application Data
(60) Provisional application No. 62/120,993, filed on Feb. 26, 2015.

(51) Int. Cl. A61N 1/36; A61M 5/172

(52) U.S. Cl. CPC A61N 1/36071; A61N 1/36139; A61M 5/1723; A61M 2230/005; A61M 2230/63

(57) ABSTRACT

A rehabilitation monitor and pain treatment assembly for a patient includes a pain treatment device adapted to be carried by a patient to administer pain treatment to the patient and an activity monitoring device adapted to be carried by the patient to monitor physical therapy of the patient. The activity monitoring device and the pain treatment device are integrated together in a single unit.

### CROSS-REFERENCE TO RELATED APPLICATIONS

This application claims priority to and the benefit of U.S. Provisional Patent Application No. 62/120,993, filed on Feb. 26, 2015, the entire contents of which are hereby incorporated by reference.

### TECHNICAL FIELD

The present invention relates generally to physical therapy and pain treatment for patients, more particularly to, a rehabilitation monitor and pain treatment assembly for a patient.

### BACKGROUND

It is known that a physical therapy routine may be implemented for remedying various symptoms suffered by a patient and typically consists of a series of physical exercises. The exercises are usually prescribed and assigned to the patient by physical therapists for rehabilitation from a specifically identified disease, injury, and/or surgical procedure. Once the physical therapy routine is prescribed, the physical therapist or exercise instructor must make sure that the patient understands how to correctly accomplish the physical exercises in the routine. To do this, the physical therapist or exercise instructor will typically demonstrate the exercises to the patient and provide written instructions for subsequent reference. Often, however, even though the patient may initially perform the entire routine, the patient may eventually disregard or not perform certain exercises in the routine over time.

Often, in conjunction with prescribing the physical therapy, physicians will prescribe a pain treatment plan in which a body worn pain treatment device is carried by the patient for applying pain treatment to the patient. It is desirable to integrate physical therapy monitoring technology with pain treatment devices without any added inconvenience or effort by the patient. Therefore, there is a need in the art to provide for monitoring rehabilitation and applying pain treatment to a patient in one assembly.

### SUMMARY

Accordingly, in one embodiment, the present invention provides a rehabilitation monitor and pain treatment assembly for a patient including a pain treatment device adapted to be carried by a patient to administer pain treatment to the patient and an activity monitoring device adapted to be carried by the patient to monitor physical therapy of the patient, wherein the activity monitoring device and the pain treatment device are integrated together.

One advantage is that a new rehabilitation monitor and pain treatment assembly is provided for a patient. Another advantage is that the rehabilitation monitor and pain treatment assembly embeds an activity monitoring device into a pain treatment device that is carried by the patient. Yet another advantage is that the rehabilitation monitor and pain treatment assembly collects data on the patient during the entire time the patient carries the assembly. Still another advantage is that the rehabilitation monitor and pain treatment assembly collects data that can be read from the assembly when the assembly is removed from the patient at the end of the pain treatment program as well as during any routine office visits during the pain treatment program. Yet a further advantage is that the rehabilitation monitor and pain treatment assembly may also include transmitting capabilities to send the data real-time during the entire pain treatment program.

Other features and advantages of the present invention will be readily appreciated, as the same becomes better understood, after reading the subsequent description taken in conjunction with the accompanying drawings.

### BRIEF DESCRIPTION OF THE DRAWINGS

FIG. 1 is a diagrammatic view of one embodiment of a rehabilitation monitor and pain treatment assembly illustrated in operational relationship with a patient.

FIG. 2 is fragmentary elevational view of the rehabilitation monitor and pain treatment assembly and patient of FIG. 1.

FIG. 3 is a diagrammatic view of the rehabilitation monitor and pain treatment assembly of FIG. 1 illustrated in operational relationship with a system.

FIG. 4 is a schematic view illustrating the rehabilitation monitor and pain treatment assembly of FIG. 1.

### DETAILED DESCRIPTION

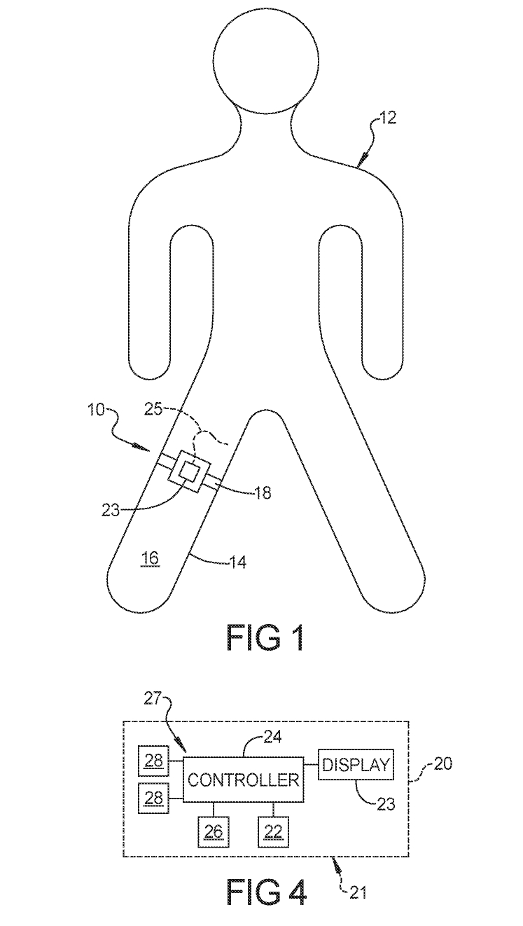

Referring to the drawings and in particular FIG. 1, one embodiment of a rehabilitation monitor and pain treatment assembly 10 is shown in operational relationship with a patient, generally indicated at 12. The patient 12 has a limb 14 and skin 16. The rehabilitation monitor and pain treatment assembly 10 is carried by the patient 12. In the embodiment illustrated in FIG. 1, the rehabilitation monitor and pain treatment assembly 10 is adapted to be installed on the limb 14 of the patient 12 to administer pain treatment to the patient 12 and to monitor physical therapy of the patient 12. It should be appreciated that the rehabilitation monitor and pain treatment assembly 10 may be installed on the limb 14 by a suitable securing or attachment device such as a strap 18. Other attachment devices are also contemplated such as a belt, wristband, armband, adhesive, tape, and the like. In some embodiments, the rehabilitation monitor and pain treatment assembly 10 may be physically embedded into the patient 12 such that the rehabilitation monitor and pain treatment assembly 10 is unable to be easily removed by the patient 12 without assistance from a medical professional.

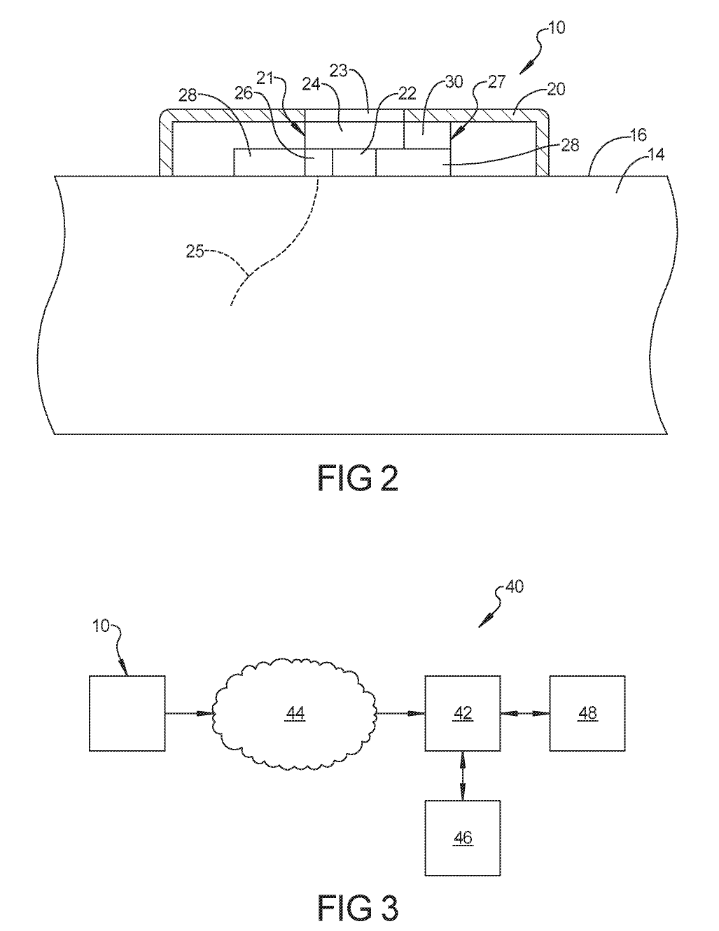

As illustrated in FIG. 2, when attached to the patient 12, the rehabilitation monitor and pain treatment assembly 10 is in contact with the skin 16 of the patient 12. The rehabilitation monitor and pain treatment assembly 10 includes a pain treatment device, generally indicated at 21, adapted to administer pain treatment to the patient 12 at a treated area. The pain treatment device 21 includes a housing 20 attached to the strap 18 (or other attachment device) and one or more sensors 22 disposed within the housing 20 for measuring at least one parameter associated with the patient. Some parameters, for example temperature, pressure, relative humidity, pH, concentration of medication/drug, concentration of salts, concentration of fatty acid on the skin 16, or other physiological parameters, can be measured from the skin 16 of the patient 12. It should be appreciated that conventional sensors can be used for obtaining these parameters.

In one embodiment, the sensors 22 may include a temperature sensor to measure the temperature on the skin surface. Temperature can be one of the parameters to determine the appropriate time to deliver treatment to the treated area based on the elevation in local skin temperature beyond a certain threshold. A thermal couple can be used with another temperature sensor for cold junction reference to the ambient environment. Another type of temperature sensor is the transistor type sensor that makes use of the fact that resistivity of the transistor changes with temperature. It should be appreciated that by monitoring the resistivity, the temperature can be deduced.

The sensors 22 may include a pressure sensor applied to the treated area. The pressure sensor may be a force sensor applied to measure pressure difference between inflamed and normal tissue. This force sensor can be made of a semiconductor material, for example silicon, which will change its resistance when a force is applied. A constant voltage is applied to measure the variation in electrical current, which reflects the force change on the contact area.

The sensors 22 may include a humidity sensor to measure the local moisture above the surface of the skin 16 when medicine is applied. This information can be used as a parameter to optimize drug delivery time, dose, and rate. A relative humidity sensor is made of a capacitor with a polymer as a dielectric material. This polymer will change its dielectric constant when absorbing moisture, hence, variation in the measured capacitance of the capacitor reflects the humidity change. This capacitance difference will be translated to an electrical voltage by a driving circuit as a measurement of humidity change. It should be appreciated that the constructions of these sensors are conventional and known in the art.

The pain treatment device 21 may include an analyzer or controller 24 for analyzing the information obtained from the sensors 22, and determining what actions from the rehabilitation monitor and pain treatment assembly 10 are required to be performed on the patient 12. The sensors 22 are in communication with the controller 24 to transmit corresponding signals to the controller 24. The controller 24 forms part of a control system. The controller 24 may be in the form of a signal processor, microcontroller or CPU with suitable analog/digital converter and signal processor. The controller 24 may have one or more microprocessors for processing instructions. Additionally or alternatively, the controller 24 may comprise one or more microcontrollers, field programmable gate arrays, systems on a chip, discrete circuitry, and/or other suitable hardware, software, or firmware that is capable of carrying out the functions described herein.

After the parameters are measured and collected by the sensors 22, the parameters will then be analyzed by the controller 24. The controller 24 will then determine appropriate pain treatment to be performed on the patient 12 according to the analysis. Temperature, pressure, and humidity are measured by the respective sensors 22 in the embodiment shown. When each of these measurements exceeds or meets the predetermined threshold or tolerance levels, the pain treatment device 21 may administer drug and/or electrical stimulus or other treatment to the affected area to sooth pain and/or reduce temperature of a swollen area from inflammation. The threshold or tolerance levels may be stored in memory that is accessible by the controller 24 for comparison to the measurements. It should be appreciated that the controller 24 may be programmed with different types of pain treatment depending on the disease and the medical history of a given patient.

The pain treatment device 21 may administer treatment to the treated area based on other factors, such as time since the last treatment, a treatment schedule, or the like. In this case, the controller 24 includes a clock and the treatment schedule is stored in the memory accessible by the controller 24. For instance, at the predetermined times for treatment, the controller 24 activates the pain treatment device 21 to administer one or more drugs and/or to provide electrical stimulus or other treatment to the appropriate area.

Additionally, the patient 12 may be able to actuate the pain treatment device 21 periodically, such as through a button on the rehabilitation monitor and pain treatment assembly 10 that actuates a switch in communication with the controller 24 so that the controller 24 causes the pain treatment device 21 to deliver treatment to the patient 12. This bolus button may only be operable periodically during treatment, e.g., once per day, and its functionality can be locked out by the controller 24 as desired.

The pain treatment device 21 may include at least one treatment source 26, or multiple treatment sources 26, which can exist in various different forms. Suitable treatment sources 26 include drug delivery devices in the form of injection devices, such as medication pumps with catheters, for delivering the drug(s) to the patient 12, and the like. Other treatment sources 26 include heaters, electrodes, magnetic field generators, optical devices (e.g., infra-red, ultraviolet), and sonic devices (e.g., ultrasound). It should be appreciated that the treatment sources 26 are conventional and known in the art.

In one embodiment, the treatment source 26 includes an implantable electrode 25 connected to the controller 24 for electrically stimulating nerves. The electrode 25 and associated controller 24 may be like those described in U.S. Patent Application Publication No. 2013/0238066, filed Sep. 6, 2012, the disclosure of which is hereby incorporated by reference. Other treatment sources that are implanted into the patient 12 are also contemplated such as catheters, and the like. In other embodiments, the treatment source 26 includes a medication delivery system with infusion tube set (also refer to numeral 25) for delivering medication to the patient 12. The medication delivery system with infusion tube set and associated controller 24 may be like those described in U.S. Pat. No. 7,048,715, filed Sep. 4, 2002, there disclosure of which is hereby incorporated by reference.

A display 23 is in communication with the controller 24 to display information relating to the pain treatment device 21 and its use. The display 23 may display the current time, the time for the next treatment, the duration of current treatment, dosages, flow rates, electrical impedance, other electrical stimulation parameters, other treatment parameters, and the like. Pushbuttons, or other input devices, may be in communication with the controller 24 to change the time on the display 23, to set dosages, treatment durations and the like, using the display 23. Some of these functions may be locked out by a medical professional once set, such as when the patient 12 receives the rehabilitation monitor and pain treatment assembly 10. In some cases, some of the input devices (e.g., buttons) are operable by the patient 12, while others are only operable by medical professionals using associated passcodes, and the like. The display 23 may be a LCD, LED, or other suitable type display. The controller 24 has the suitable software and hardware, e.g., drivers, etc. necessary for displaying graphical images, messages, and the like on the display 23.

The rehabilitation monitor and pain treatment assembly 10 also includes an activity monitoring device, generally indicated at 27, to monitor physical therapy of the patient 12. The activity monitoring device 27 may include one or more sensors 28 for monitoring motion of the patient 12. The required number and location of the sensors 28 varies based on the characteristics of the therapy for the patient 12. Placing the sensors 28 on points where motion is greatest results in the clearest signal but may require additional sensors, wiring, or electronics. The sensors 28 may include stand-alone signal processing and memory capability. In the embodiment shown, the activity monitoring device 27 shares the same controller 24 as the pain treatment device 21. In this case, the sensors 28 generate and transmit associated sensor signals to the controller 24, which can then display on the display 23 or store in the memory information related to the activities being performed by the patient 12, such as duration of activity, type of activity, number of steps walked by the patient (e.g., when the sensors 28 function as a pedometer), etc.

The sensors 28 can be configured to provide data related to certain activities, exercise or movements performed by the patient 12. The sensors 28 can be configured to sense various movements related to activities, exercises or movements that are part of the patient's 12 therapy plan. It should be appreciated that one or more sensors 28 can be used depending upon the activities, exercises and movements performed.

The sensors 28 may be of various types and combinations of sensors such as motion sensors, inertial sensors, strength sensors, physiologic sensors such as heart rate sensors, blood pressure sensors, skin conductance or perspiration rate sensors, temperature sensors, pain sensors and oxygen sensors, etc., e.g., for detecting blood oxygen level, etc.

The rehabilitation monitor and pain treatment assembly 10 may include one or more wireless transmitters or transceivers 30 configured to transmit or transmit/receive wireless signals of data. The wireless transmitters or transceivers 30 may be in communication with the controller 24 to transmit/receive data to/from the controller 24 and/or to/from the memory accessible by the controller 24. The transmitters or transceivers 30 can, for example, be Radio Frequency (RF) transmitters or Infrared transmitters. Moreover, the transmitters or transceivers 30 can be configured to operate in accordance with any of a plurality of communication protocols that are collectively referred to as WiFi standards, WiGig standards, and UltraWideband Standards. The transmitters or transceivers 30 can also comply with the ZigBee standard, Bluetooth, and in particular the low power Bluetooth standard, the IRDA standard, various standards or protocols that make use of the industrial, scientific, and medical (ISM) radio band, short-range device (SRD) bands, the European SRD bands, the Chinese WPAN bands as well as various other low power standards designed for short range communication. It should be appreciated that the transmitters or transceivers 30 may be part of the pain treatment device 21 and/or the activity monitoring device 27.

It should also be appreciated that the pain treatment device 21 and activity monitoring device 27 are integrated into a single unit or assembly. As a result, for instance, the activity monitoring device 27 cannot be removed by the patient 12, without also removing the pain treatment device 21, since they are integrated into the same unit or assembly, and in some cases, like the embodiment shown in FIG. 2, integrated into the same housing and sharing the same controller 24. In some cases, because the pain treatment device 21 is implanted in the patient 12, or includes one or more treatment sources that are implanted in the patient 12 (e.g., catheter or electrode), the pain treatment device 21 cannot be removed without assistance from a physician or other medical professional at a medical facility, such as a clinic or hospital. Accordingly, the activity monitoring device 27 also cannot be removed without assistance from a physician or other medical professional. This helps to avoid situations in which the patient 12 may otherwise choose to simply remove the activity monitoring device 27, such as when the activity monitoring device 27 is a standalone device.

Referring to FIG. 3, the rehabilitation monitor and pain treatment assembly 10 may be used in a system, generally indicated at 40, that includes a server 42 that can be interfaced with the rehabilitation monitor and pain treatment assembly 10 via a network 44, such as the Internet. The server 42 can include multiple components and resources. For example, the server 42 can include multiple servers for redundancy and to carry out various functions. The server 42 can also include multiple data base servers, routers, network interfaces, and multiple processors as required. The server 42 can in general include all of the hardware and software resources needed to perform the processes described herein. It should be appreciated that the network 44 can include one or more wired or wireless PANs, LANs, WANs, MANs etc., interfaced as required to enable the communication described herein.

The data from the sensors 28 can also be used by the server 42 to determine outcome measurement of the success or progress of therapy or treatment, whether in the area of physical therapy, wellness, etc. This type of outcome measurement can be referred to as a “function determination”. Often this will involve data related to strength. Thus, the sensors 28 can include strength sensors configured to transmit data to the server 42 for use in function determination, e.g., the effectiveness of certain exercise in terms of improved strength.

The function determination can be more complex than simple strength determinations, or determinations that include angle, speed, etc. For example, the function determination can be designed to determine whether an individual can function the way they did before an injury, or whether they are still limited. As such the function determination can involve multiple sensor types, including GPS sensors, physiologic sensors, strength sensors, motion sensors, inertial sensors, etc. For example, the sensors 28 may include one or more 3-dimensional accelerometers for measuring acceleration of the particular area of focus in the physical therapy, such as leg acceleration. The accelerometer transmits a signal to the controller 24, which then utilizes its one or more processors to calculate acceleration values over time for the patient 12 and compare those values to a threshold value stored in the memory of the rehabilitation monitor and pain treatment assembly 10. The processor(s) can then compare the calculated acceleration values to the threshold value and determine whether any of the calculated accelerations exceed the threshold value, which may be indicative of restored function. The results may be shown on the display 23, which is integrated in the rehabilitation monitor and pain treatment assembly 10 and controlled by the controller 24, and/or the results may be stored in the memory for later retrieval.

The server 42 can then determine progress, performance levels, and compliance with and completion of a physical therapy plan. The server 42 can also generate messages for the patient 12 or another individual or, e.g., a coach, physician, etc., and can determine whether the patient 12 is ready to advance in level or stage, etc., within the physical therapy plan. The server 42 can also determine whether the physical therapy plan needs to be updated or changed in other ways as well. By virtue of the connection between the rehabilitation monitor and pain treatment assembly 10 and the server 42, messages can be sent to the rehabilitation monitor and pain treatment assembly 10 via the transceivers 30 and can be used by the controller 24, for instance to update the treatment schedule, e.g., times for treatment, or messages can be displayed to the patient on the display 23.

The system 40 may include a storage system 46 where data gathered via the rehabilitation monitor and pain treatment assembly 10 and transmitted to server 42 can be stored. Various algorithms and routines resident on or available to the server 42 can be configured to then automatically generate reports, determine therapy compliance, make function determinations, determine progress and outcomes as well as to generate new plans or modify plans. It should be appreciated that the system 40 may generate certain reports that can be made available to a healthcare provider 48 interacting with the server 42, e.g., physical therapist, occupational therapist, physician, personal trainer, coach, wellness expert, etc. The healthcare provider 48 can interact with the server 42 using a network such as the Internet, wired or wireless connections, etc. The healthcare provider 48 may receive alerts, notifications, or other messages relating to the patient 12 and their progress with respect to the physical therapy plan.

Under the control of the controller 24, the rehabilitation monitor and pain treatment assembly 10 can be configured to scan multiple sensors 22 and 28 and receive data therefrom. The rehabilitation monitor and pain treatment assembly 10 can also be configured to aggregate and store the data. The rehabilitation monitor and pain treatment assembly 10 can be configured to not only aggregate the data but to also correlate the data, e.g., from the different sensors 22 and 28, based on time stamps or other information included in the data. It should be appreciated that the data can be read from the rehabilitation monitor and pain treatment assembly 10 to determine if the patient 12 is completing the therapy prescribed.

In operation, the patient 12 can be outfitted with the rehabilitation monitor and pain treatment assembly 10 to monitor the activities, exercises and movements they perform while also enabling pain treatment. The sensors 28 can then communicate data related to the activities, exercises and movements, as well as other types of data, to the server 42 where it can be stored and analyzed and where reports and messages can be generated, treatment plans assessed and modified as needed. It should also be appreciated that the rehabilitation monitor and pain treatment assembly 10 can treat pain at the location the rehabilitation monitor and pain treatment assembly 10 is installed on the patient 12.

The present invention has been described in an illustrative manner. It is to be understood that the terminology, which has been used, is intended to be in the nature of words of description rather than of limitation. Many modifications and variations of the present invention are possible in light of the above teachings. Therefore, the present invention may be practiced other than as specifically described.

What is claimed is:

1. A rehabilitation monitor and pain treatment assembly for a patient comprising:
a pain treatment device adapted to be carried by a patient to administer pain treatment to the patient; and
an activity monitoring device adapted to be carried by the patient to monitor physical therapy of the patient,
wherein said activity monitoring device and said pain treatment device are integrated together.

2. A rehabilitation monitor and pain treatment assembly as set forth in claim 1 including a display.

3. A rehabilitation monitor and pain treatment assembly as set forth in claim 1 wherein said pain treatment device comprises a treatment source.

4. A rehabilitation monitor and pain treatment assembly as set forth in claim 3 wherein said treatment source is configured to be implanted in the patient.

5. A rehabilitation monitor and pain treatment assembly as set forth in claim 3 wherein said treatment source includes an electrode.

6. A rehabilitation monitor and pain treatment assembly as set forth in claim 3 wherein said treatment source includes a catheter.

7. A rehabilitation monitor and pain treatment assembly as set forth in claim 1 wherein said pain treatment device includes at least one sensor for measuring at least one parameter of the patient.

8. A rehabilitation monitor and pain treatment assembly as set forth in claim 7 wherein said at least one sensor comprises at least one of a temperature sensor, pressure sensor, or humidity sensor.

9. A rehabilitation monitor and pain treatment assembly as set forth in claim 7 wherein said pain treatment device includes a controller for analyzing information obtained from said at least one sensor.

10. A rehabilitation monitor and pain treatment assembly as set forth in claim 9 wherein said pain treatment device is configured to deliver treatment to the patient based on the analyzed information.

11. A rehabilitation monitor and pain treatment assembly as set forth in claim 1 wherein said activity monitoring device includes at least one sensor for sensing motion of the patient.

12. A rehabilitation monitor and pain treatment assembly as set forth in claim 11 wherein said at least one sensor includes an accelerometer.

13. A rehabilitation monitor and pain treatment assembly as set forth in claim 11 wherein said activity monitoring device includes at least one transmitter for transmitting data associated with said at least one sensor.

14. A rehabilitation monitor and pain treatment assembly as set forth in claim 1 including a housing, said pain treatment device and said activity monitoring device being disposed within said housing.
